(19) United States
(12) Patent Application Publication
(10) Pub. No.: US 2010/0297648 A1
(43) Pub. Date: Nov. 25, 2010

(54) METHOD FOR DIAGNOSING SUSCEPTIBILITY TO POST-TRAUMATIC SCAR-TISSUE FORMATION

(76) Inventors: Márta Széll, Szeged (HU); László Rovó, Szeged (HU); Lajos Kemény, Szeged (HU)

Correspondence Address: HAHN & VOIGHT PLLC, 1012 14TH STREET, NW, SUITE 620, WASHINGTON, DC, 20005, US

(21) Appl. No.: 12/744,988

(22) PCT Filed: Apr. 28, 2008
(86) PCT No.: PCT/IB08/51627

§ 371 Date: Aug. 10, 2010

(30) Foreign Application Priority Data
Apr. 27, 2007 (HU) P0700313

(51) Int. Cl. C12Q 1/68

(52) U.S. Cl. 435/6

(57) ABSTRACT

Disclosed is an in vitro method for diagnosing susceptibility to post-traumatic scar tissue formation, wherein from a biological sample of a patient the nucleotide of the -509 position of the TGF-β1 gene is determined and if said -509 position contains exclusively C, thus it is the homozygotic wild type allele, then said patient is considered to be susceptible to post-traumatic scar tissue formation. The invention relates furthermore to diagnostic kits for the detection of susceptibility to post-traumatic scar tissue formation, preferably for the detection of susceptibility to tracheal stenosis from a biological sample.

### FIELD OF THE INVENTION

The invention disclosed herein relates to an in vitro method for diagnosing susceptibility to post-traumatic scar tissue formation, wherein from a biological sample of a patient the nucleotide of the -509 position of the TGF-β1 gene is identified and if said -509 position contains exclusively C, thus it is the homozygotic wild type allele, then said patient is considered to be susceptible to post-traumatic scar tissue formation. The invention relates furthermore to diagnostic kits for the detection of susceptibility to post-traumatic scar tissue to formation, preferably for the detection of susceptibility to tracheal stenosis from a biological sample.

### BACKGROUND OF THE INVENTION

The air goes through the larynx and the trachea to the lungs so an emerging stenosis or atresia in this tract can cause asphyxia, a sufficient declining of the quality of life, and in more serious cases it can even lead to abhorrent life conditions. One frequent form of the stenosis is the evolving of the intra-laminar scar-tissues due to the damages of the anatomic formulas. Until the middle of the 20^{th} century, the stenosis of the upper respiratory tract was developed mostly as the result of an exterior cervical trauma, infectious diseases (e.g. diphtheria, syphilis, etc,) causing cicatricose. However, during the recent decades, this pathologic condition is mostly the result of complications caused by long-lasting artificial respiration via intubations due to traffic accidents, in the complex routine operational procedures based on the intensive care, which in societies having well-developed health care services can touch a considerable proportion of the population. The cuff of the respiration tube can cause decubitus on the mucous membrane, which is according to the literature data can lead to the forming of a scar-tissue and granulation in 1-3 percentage of the long-term intubation cases and results in a stenosis with breathing difficulties. There are several well-known solutions to eliminate the developed suffocating, but nowadays the best results can be ensured by means of expensive operations made by external surgical procedure which means for the patient a further physical and mental burden and very often requires a long-lasting healing period.

The characteristic appearance of the post-traumatic scar-tissue formation is the post-intubation cicatricose stenosis. The development of such stenosis depends on several factors in which—according to our current knowledge—the method of performing and the duration of intubation, the micro-circulation of the involved tissue play the cardinal role. However, recent publications support that during the intensive therapy the following solutions can affect favorably the development rate of the stenosis: more tolerable respiratory tubes [Sengputa P. et al. 2004; Dullenkopf A. et al. 2003], drugs decreasing the gastroesophagial reflux and blocking the proton-pump [Roh J. L. et al. 2006, Koufman J. A., 1991], the local application of preparations inhibiting fibroblast proliferation [Lorenz R. R., 2003, Roh J. L. et al. 2007, Simpson C. B. and James J. C., 2006] etc. In order to preserve the function of the larynx it is also a very important question to define the necessity and the time of the tracheotomy instead of the trans-laryngeal respiration. By means of the methods mentioned above the threat of the forming of the stenosis in the respiratory tract can be reduced, but all of these will increase the to expenses of intensive therapy.

According to clinical observations, there are differences in patients treated with similar intubation circumstances concerning the frequency of forming of stenosis. It can induce the possibility that the development rate of the scar-tissues, similarly to the cheloid formations examined in dermatology, can vary and can be explained by the individual differences, characteristics of the regeneration mechanism and the genetic background. Today, knowing the appropriate marker gene and using a quick, inexpensive and routine molecular-genetic examination, the patients belonging to the risk-group could be separated, helping this way the decision regarding the adequate, cost-effective therapy during intensive care. The expected reduction in the number of the operational interventions due to the post-intubation stenosis could result additional cost reductions.

Based on the above arguments, the aim of the elaboration of the present invention was to find a genetic marker which can be easily identified and whose presence forecasts with high probability the development of the most frequent form of the post-traumatic scar-tissue, the post-intubation stenosis in the respiratory tract, the tracheal stenosis.

An investigation into the international literature directed our attention to the polymorphisms of transforming growth factor-β1 (TGF-β1), a gene which plays a role in the formation of the various fibrotic disorders of the respiratory tract. [Wu L. et al., 2004, Celedon J. C., et al., 2004, Lawson W. E. and Loyd J. E. 2006, Drumm M. L., et al., 2005, Sheppard D. 2006, Olman M. A. 2003, Shah R. et al., 2006, Barnes P. J. 2004]. It was also confirmed, that the polymorphisms can be either susceptibility or protective factors in various pathologic conditions. Two of the TGF-β1 polymorphisms are located in the protein coding region (codon 10 and codon 25), and two of them are located in the promoter region (-800 G/A and -509 C/T). No data was found in the literature indicating that these polymorphisms could play any role in the pathogenesis of the post-traumatic tracheal scar-tissues.

### SUMMARY OF THE INVENTION

The solution according to the present invention is based on the surprising and unexpected finding, that the ratio of the C/C genotype of -509 C/T TGF-β1 polymorphisms is higher among patients with post-traumatic tracheal scar-tissue. Therefore the determination of said polymorphism creates a good possibility to forecast susceptibility to the development of the post-traumatic tracheal scar-tissue.

Therefore present invention relates to an in vitro method for diagnosing susceptibility to post-traumatic scar tissue formation, said method comprising the steps of

(a) DNA containing samples are isolated from a patient and a population of fragments comprising the nucleotide at position -509 of the transforming growth factor-β1 (TGF-β1) gene is amplified;

(b) the nucleotide at position -509 of the TGF-β1 gene is identified in the amplified population of fragments; and

(c) said patient is considered susceptible to post-traumatic scar tissue formation if said sample contains exclusively C at said -509 position indicating thereby the presence of a homozygotic wild type allele.

In a preferred embodiment of the present invention in the above step (a) said DNA fragment is amplified from a blood sample of said patient, preferably by a PCR method.

In a further preferred embodiment of the present invention the following primers are used for the amplification of the gene region harboring said -509 position:

| | |
|---|---|
| GGAGAGCAATTCTTACAGGTG | (Seq. ID No 1.) |
| and | |
| TAGGAGAAGGAGGGTCTGTC. | (Seq. ID No 2.) |

According to a further preferred embodiment of the present invention in step (b) of the above method said nucleotide base is identified by using an RFLP method, preferably DdeI restriction enzyme is used in said RFLP method.

In a still further preferred embodiment of the present invention in step (b) of the above method said nucleotide is identified by using any method based on sequencing or hybridization that is suitable for the detection of mismatches in nucleotide base paring.

According to a still further preferred embodiment of the present invention the susceptibility to post-traumatic scar tissue formation is a susceptibility to tracheal stenosis.

The invention further relates to a diagnostic kit for in vitro detection of susceptibility to post-traumatic scar tissue formation from a biological sample, said kit comprising:

(a) primers that can specifically bind to sequences being in suitable distances in both 5′ and 3′ direction from said -509 position of said TGF-β1 gene;

(b) instructions for performing a method for diagnosing susceptibility to post-traumatic scar tissue formation; and optionally

(c) reagents for performing a method according to any one of claims 1 to 8.

In a preferred embodiment of the present invention the above diagnostic kit comprises the following primers:

| | |
|---|---|
| GGAGAGCAATTCTTACAGGTG | (Seq. ID No 1.) |
| and | |
| TAGGAGAAGGAGGGTCTGTC. | (Seq. ID No 2.) |

### BRIEF DESCRIPTION OF THE DRAWING

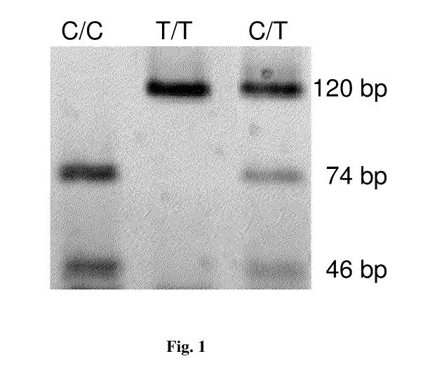

FIG. 1 shows the genotyping of the amplified DNA-fragment followed after digestion by the DdeI restriction endonuclease on an agarose gel. The fragments appearing on the gel are the followings:
46+74 bp fragments: C/C genotype,
single 120 bp fragment: T/T genotype and
120+74+46 bp fragments: C/T genotype.

### DETAILED DESCRIPTION OF THE INVENTION

In the detailed description below, we shall demonstrate several examples concerning the method according the present invention and concerning the diseases, which can be diagnosed by performing the method according to the present invention, etc. However it is obvious for a person skilled in the art that only certain embodiments of the present invention are described to assist an artisan. Clearly we have no intention to limit the scope of the present invention with the described examples, they are only assisting in the use of the present invention.

During the diagnostic procedure of the present invention to identify said polymorphism, PCR-RFLP process can be preferably used, which essentially consists of the amplification of the gene region with the polymorphism to be examined (PCR), the digestion by a restriction endonuclease and the analysis of the resulted fragment (RFLP). We have to emphasize that the procedure according to the present invention will not be limited to the above PCR-RFLP process. The polymorphism can also be identified by other known methods, like those, which are based on the identification of the nucleic acid sequence or on hybridization and other processes based on the detection of improper base-pairing. This process can be for example a PCR process and the subsequent sequencing, or a PCR-reaction with an allele-specific TaqMan test.

According to one of the preferred embodiments of the present invention, the genetic factor—firstly ever indicated for the susceptibility to the development of post-traumatic tracheal scar-tissue—can be determined in a modest molecular biology laboratory, equipped minimally with the following instruments:

PCR device for the amplification of the gene fragment harboring said polymorphism,

37° C. thermostat for the restriction endonuclease digestion,

gel electrophoresis device for separation of the fragments on an agarose gel, and

gel documentation system for the photography of the agarose gel, evaluation of results and data recording.

One of the preferred embodiments of the present invention can be performed by the following way: genomic DNA-isolation (approx. 1 hour) is performed from 2-3 ml venous blood taken from the patient before intubation; amplification of the gene region harboring the polymorphism by PCR (approx. 2-3 hours) with the following primers:

| | |
|---|---|
| TGFB509F: GGAGAGCAATTCTTACAGGTG | (Seq. ID No 1.) |
| and | |
| TGFB509R: TAGGAGAAGGAGGGTCTGTC; | (Seq. ID No 2.) |

digestion of the amplified DNA-fragment by the DdeI restriction enzyme (approx. 2 hours), genotyping on 5% agarose gel, separating the low molecular weight fragments with high efficiency (approx. 2 hours).

The fragments appearing on the agar gel are the following:

46+74 bp fragments: C/C—it indicates, that the given individual is wild type at the -509 position of the TGF-β1 gene

one 120 bp fragment: T/T—it indicates, that the given individual is a homozygote mutant at the -509 position of the TGF-β1 gene

120+74+46 bp fragments: C/T—it indicates, that the given individual is a heterozygote at the -509 position of the TGF-β1 gene.

Therefore it is obvious that genotyping of the -509C/T TGF-β1 position of a patient can be done within one working day. In case an intubated patient harbors the C/C wild genotype, indicating susceptibility to tracheal stenosis, the physician has the possibility to make the proper decision by which the risk of stenosis formation can be reduced.

### EXAMPLES

The following example further illustrates the present invention but should not be construed as in any way limiting its scope.

### Example 1

At the Department of Oto-Rhino-Laryngology and Head-Neck Surgery in cooperation with the Department of Dermatology and Allergology, (both at the University of Szeged, Hungary) we performed the following experiment: 66 patients were enrolled to the study from which 30 patients had no reaction concerning the development of the post-traumatic tracheal stenosis after the intensive care intubation; while in case of 36 patients, the above mentioned pathologic condition developed. Patients' data are summarized in Table 1.

TABLE 1

| | | Average duration |
|---|---|---|
| | Average age | of the intubation |
| | (years ± SD) | (days ± SD) |
| Patients having tracheal stenosis | 44.63 ± 18.30 | 7.87 ± 4.92 |
| n = 36 | | |
| Control population | 62.07 ± 16.44 | 6.93 ± 5.32 |
| n = 30 | | |

We took venous blood from the patients and purified genomic DNA and compared the prevalence of the four TGF-β polymorphisms known in the literature in the two patient groups. To test the four polymorphisms we used a simple molecular biology method, which was the above mentioned PCR-RFLP.

In accordance with our experimental results, one of the tested polymorphisms—the -509C/T—showed significant difference in the allele distribution between the two patient groups, namely in the group where despite the prolonged intubation the tracheal stenosis did not develop, the ratio of the heterozygotes (C/T) was much higher at said position, while in case of patients where tracheal stenosis formed as a result of the intubation, the ratio of the heterozygotes was much lower.

TABLE 2
The distribution of the promoter polymorphism genotype TGF-
β1 gene −509 C/T between patient groups having tracheal
stenosis (TS; n = 36) and the control population (C; n = 30)

| | | | | Genotypes in the |
|---|---|---|---|---|
| | The number of the patients and their | | | examined total |
| | ratio (%) having the given genotype | | | population |
| Genotypes | TS | C | | TS + C |
| C/C | 21 (58.3%) | 7 (23.3%) | p = 0.0116 | 28 (42.4%) |
| C/T | 14 (38.8%) | 21 (70%) | OR: 4.5 | 35 (53%) |
| T/T | 1 (2.7%) | 2 (6.6%) | | 3 (4.5%) |
| Total | 36 (100%) | 30 (100%) | | 66 (100%) |

The processing of the data (Table 2)—calculation of the so called Odds ratio (OR)—showed that the individuals being homozygote wild types at the given position had 4.5-times higher chance regarding the development of the tracheal stenosis than the other individuals who were heterozygotes on this position. Our test results did not include the homozygote mutant individuals because the literature data as well as our assay showed that this genotype is rare within the Caucasian population. The above mentioned denotes that the genotyping of the TGF-β1 -509C/T polymorphism of the patients treated by intubation can help in the to estimation of susceptibility to tracheal stenosis. Our assumption is particularly supported by the fact that according to the internet databases as well as by our data, half of the Caucasian population is homozygote wild type while the other half is heterozygote for the given allele. Accordingly, half of the population has four-times higher chance to develop post-intubation tracheal stenosis due to the intensive therapy than the other half of the population. The presented results make it possible to perform an easy and simple diagnostic procedure, which can select the above mentioned risk group.

Our result raise the possibility, that the TGF-β1 gene and/or the TGF-β1 protein coded by it can be introduced in future as a therapy target in the treatment of scarry tracheal stenosis.

### REFERENCES

1. Sengupta P., et al. Endotracheal tube cuff pressure in three hospitals, and the volume required to produce an appropriate cuff pressure. BMC Anesthesiol. 2004; 29;4(1):8.

2. Dullenkopf A., et al. Fluid leakage past tracheal tube cuffs: evaluation of the new Microcuff endotracheal tube. Intensive Care Med. 2003; 29(10):1849-53.

3. Roh J. L., et al. Effect of acid, pepsin, and bile acid on the stenotic progression of traumatized subglottis. Am J Gastroenterol. 2006; 101(6):1186-92.

4. Koufman J. A. The otolaryngologic manifestations of gastroesophageal reflux disease (GERD): a clinical investigation of 225 patients using ambulatory 24-hour pH monitoring and an experimental investigation of the role of acid and pepsin in the development of laryngeal injury. Laryngoscope. 1991; 101(4 Pt 2 Suppl 53):1-78.

5. Lorenz R. R. Adult laryngotracheal stenosis: etiology and surgical management. Curr Opin Otolaryngol Head Neck Surg. 2003; 11(6):467-72.

6. Roh J. L., et al. Benefits and risks of mitomycin use in the traumatized tracheal mucosa. Otolaryngol Head Neck Surg. 2007; 136(3):459-63.

7. Simpson C. B. and James J. C. The efficacy of mitomycin-C in the treatment of laryngotracheal stenosis Laryngoscope. 2006; 116(10):1923-5.

8. Wu L., et al. Transforming growth factor-beta1 genotype and susceptibility to chronic obstructive pulmonary disease. Thorax 2004; 59: 126-129.

9. Celedon J. C., et al. The transforming growth factor-beta1 (TGFB1) gene is associated with chronic obstructive pulmonary disease (COPD). Hum Mol Genet 2004; 13: 1649-1656.

10. Lawson W. E. and Loyd J. E. The genetic approach in pulmonary fibrosis: can it provide clues to this complex disease? Proc Am Thorac Soc 2006; 3: 345-349.

11. Drumm M. L., et al. Genetic modifiers of lung disease in cystic fibrosis. N Engl J Med 2005; 353: 1443-1453.

12. Sheppard D. Transforming growth factor beta: a central modulator of pulmonary and airway inflammation and fibrosis. Proc Am Thorac Soc 2006; 3: 413-417.

13. Olman M. A. Epithelial cell modulation of airway fibrosis in asthma. Am J Respir Cell Mol Biol 2003; 28: 125-128.

14. Shah R., et al. Allelic diversity in the TGFB1 regulatory region: characterization of novel functional single nucleotide polymorphisms. Hum Genet 2006; 119: 61-74.

15. Barnes P. J. Mediators of chronic obstructive pulmonary disease. Pharmacol Rev 2004; 56: 515-548.

1. In vitro method for diagnosing susceptibility to tracheal stenosis, comprising the steps of
(a) DNA containing samples are isolated from a patient and a population of fragments comprising the nucleotide at position -509 of the transforming growth factor-β1 (TGF-β1) gene is amplified;
(b) the nucleotide at position -509 of the TGF-β1 gene is identified in the amplified population of fragments; and
(c) said patient is considered susceptible to post-traumatic scar tissue formation if said sample contains exclusively C at said -509 position indicating thereby the presence of a homozygotic wild type allele.

2. The method according to claim 1, wherein in step (a) said DNA fragment is amplified from a blood sample of said patient.

3. The method according to claim 1, wherein in step (a) a PCR method is used for the amplification of said population of fragments of the TGF-β1 gene.

4. The method according to claim 3, wherein the following primers are used for the amplification of the gene region harboring said -509 position:

| | |
|---|---|
| GGAGAGCAATTCTTACAGGTG | (Seq. ID No 1.) |
| and | |
| TAGGAGAAGGAGGGTCTGTC. | (Seq. ID No 2.) |

5. The method according to claim 1, wherein in step (b) said nucleotide base is identified by using an RFLP method.

6. The method according to claim 5, wherein DdeI restriction enzyme is used in said RFLP method.

7. The method according to claim 1, wherein in step (b) said nucleotide is identified by using any method based on sequencing or hybridization that is suitable for the detection of mismatches in nucleotide base paring.

8. (canceled)

9. Diagnostic kit for in vitro detection of susceptibility to tracheal stenosis from a biological sample, said kit comprising:
(a) primers that can specifically bind to sequences being in suitable distances in both 5′ and 3′ direction from said -509 position of said TGF-β1 gene;
(b) instructions for performing a method for diagnosing susceptibility to tracheal stenosis; and optionally
(c) reagents for performing a method according to claim 1.

10. The diagnostic kit according to claim 9 comprising the following primers:

| | |
|---|---|
| GGAGAGCAATTCTTACAGGTG | (Seq. ID No 1.) |
| and | |
| TAGGAGAAGGAGGGTCTGTC. | (Seq. ID No 2.) |


SEQUENCE LISTING (2 sequences)

SEQ ID NO: 1 - DNA, 21 bp, Artificial
  Primer
ggagagcaattcttacaggtg

SEQ ID NO: 2 - DNA, 20 bp, Artificial
  Primer
taggagaaggagggtctgtc